(19) United States
(12) Patent Application Publication
(10) Pub. No.: US 2010/0074851 A1
(43) Pub. Date: Mar. 25, 2010

(54) COSMETIC OR PHARMACEUTICAL COMPOSITION CONTAINING HYALURONIC ACID

(75) Inventors: Jacques Dubois, Villers-la-Ville (BE); Alfred Marchal, Waterloo (BE)

Correspondence Address: MERCHANT & GOULD PC, P.O. BOX 2903, MINNEAPOLIS, MN, 55402-0903, US

(73) Assignee: Auriga International S.A., Braine-l'alleud (BE)

(21) Appl. No.: 12/375,857

(22) PCT Filed: Aug. 1, 2007
(86) PCT No.: PCT/EP2007/057987

§ 371 Date: Oct. 21, 2009

Related U.S. Application Data
(60) Provisional application No. 60/841,656, filed on Aug. 30, 2006.

(30) Foreign Application Priority Data
Aug. 1, 2006 (EP) 06118270.5

(51) Int. Cl. A61K 31/728; A61K 8/73; A61P 17/00; A61P 17/02; A61P 17/10; A61Q 19/08; A61Q 17/04

(52) U.S. Cl. 424/59; 514/54

(57) ABSTRACT

A cosmetic or pharmaceutical composition includes particles of hyaluronic acid having a molecular weight between 20000 Da and 3000000 Da, combined with a polyamine.

### FIELD OF THE INVENTION

The present invention is related to a (preferably colloidal) cosmetic or pharmaceutical composition, comprising hyaluronic acid particles, preferably made of hyaluronic acid molecules having a high molecular weight and polyamines.

The present invention is also related to this composition for the treatment of wound healing and as skin wrinkles or lips filler.

### BACKGROUND OF THE INVENTION AND STATE OF THE ART

The hyaluronic acid (also referred as HA or HLA) is a naturally occurring polysaccharide that consists of Na acetyl d-glucosamin and β-glucocoronic acid.

The unique visco-elastic nature of hyaluronic acid along with its biocompatibility and non-immunogenicity has led to its use in a number of clinical applications which include:

supplementation of joined fluids in arthritis,

surgical aid in eye surgery and,

healing and regeneration of surgical wounds.

Applications of hyaluronic acid containing cosmetic are proposed to moisturize and restore elasticity thereby achieving an anti-wrinkle effect. Hyaluronic acid base composition formulation or sunscreen is also used to protect the skin against UVA radiations due to the free radicals binding properties of the hyaluronic acid.

It has also been reported that injection of hyaluronic acid (either in a stabilised form or in combination with other polymers) into the dermis, can reduce facial line and wrinkles in the long term with fewer side effects and better tolerability compared with the use of collagen.

However, this injection could present some side effects, such as allergic reactions possibly due to impurities present in the extracted hyaluronic acid.

Furthermore, it has been also used as a drug delivery agent through various routes of administration (including ophthalmic, nasal, pulmonary, parenteral and topical).

Topical delivery for the treatment of skin disorders offers numerous potential advantages over system therapy, such as those involving the use of oral or parenteral products.

However, the targeted delivery of drugs for the treatment of topical disorders is not trivial.

The physiological function of the stratum corneum, the outer most and none-viable layers of the skin, acts as protective barriers for the body and as such, it is particularly effective at preventing the permeation of hydrophilic molecules, including such drugs into deeper skin layers where viable cells are located.

Therefore, additional methods based upon chemical or physical penetration enhancement are often required to traverse this barrier.

Consequently, in topical applied formulation, there is a need for a system that helps the drugs to negotiate successively the stratum corneum, then avoid further penetration all the way across the deep layer of the skin (the dermis) and into the blood strain.

Obviously, this requires a final balance system that is not easily achieved.

U.S. Pat. No. 5,571,503 describes anti-pollution cosmetic composition which comprise water, and emulsified and dispersed the water, an antipollution complex, an anti-free radical complex, a sunscreen, and a micellar complex comprising: phospholipids, glycosphingolipids, panthenol, cholesterol, Crataegus extract and sodium hyaluronate. This composition is advantageously used in UV-A/UV-B protection of skin.

U.S. Pat. No. 5,879,684 describes skin tightening formulation and method for treating skin. This composition comprises a skin tautening aqueous gel comprising an homogeneous blend of water, a dispersed finely particulate vegetable based tensor agent containing a protein-base complex of vegetable extract and polysaccharides, a polymeric gelling agent, a liquid hydrocarbon dispersing aid and a non-ionic surfactant that maintains the dispersion of the liquid hydrocarbon dispersing aid in the gel. This homogeneous blend may also comprise hyaluronate 2% to 7% (by weight) of sodium.

The European patent application EP1 407 759 describes a cosmetic composition in colloidal form comprising hyaluronic acids and chitosan. This cosmetic composition has a moisturizing activity and is useful for helping the elasticity of the skin and may further contain a mixture of hyaluronic acids having different molecular weights in an amount of 5 to 25% (by weight, compared to the total weight of the composition) a cosmetically effective amount of chitosan and cosmetically acceptable diluents and preservatives.

The molecular weight of hyaluronic acids particles are low and comprised in the range of 400 to 4000 daltons.

The US patent application US 2004/0076680 describes a complex made of hyaluronic acid derivative that could be used to fill wrinkles.

### AIMS OF THE INVENTION

The present invention aims to provide a new hyaluronic cosmetic composition and/or pharmaceutical composition which does not present the drawbacks of the state of the art and presents improved characteristics, in particular in topical administration, or could be used as a carrier of various active cosmetic and/or therapeutical compounds.

Another aim of the present invention is to provide such cosmetic and/or pharmaceutical composition comprising hyaluronic acid combined with improved topical administration characteristics.

A further aim of the present invention is to obtain a cosmetic and/or pharmaceutical composition that improves the visco-elastic properties of human skin (improves moisture and elasticity of the skin) and could be used as an anti-wrinkles filler or as lips filler.

A further aim of the present invention is to provide such composition for the treatment of wound healing.

A further aim of the present invention is to provide such composition having reduced or no side effects and having improved tolerability for human skin.

A last aim of the present invention is to provide such composition which is capable of protecting human skin against ultraviolet (UVA) radiations, but also protects human skin against deleterious effects of free-radicals.

### SUMMARY OF THE INVENTION

The present invention is related to a (preferably colloidal) cosmetic and/or pharmaceutical composition comprising (a polydispersed composition made of) particles of hyaluronic acid molecules having a high molecular weight in combination with one or more polyamines, preferably putrecine.

The inventors have observed unexpectedly that such composition comprising particles of hyaluronic acid molecules having a high molecular weight (one monomeric unit of hyaluronic acid has a molecular weight of 401,329 Da) could be advantageously used (as such) as a cosmetic or pharmaceutical composition, because these hyaluronic acid molecules of high molecular weight (higher than 20000 daltons (Da), but lower than 3000000 daltons (Da)) present an advantageous penetration profile and cosmetic or therapeutical properties defined by their molecular size.

Preferably, the hyaluronic acid molecules present in the composition according to the invention present a molecular weight higher than 30000 daltons (Da), 40000 daltons (Da), 50000 daltons (Da), 60000 daltons (Da), 70000 daltons (Da), 80000 daltons (Da), 90000 daltons (Da), 100000 daltons (Da), 150000 daltons (Da), 200000 daltons (Da), 250000 daltons (Da), 300000 daltons (Da), 350000 daltons (Da), 400000 daltons (Da), 500000 daltons (Da), 600000 daltons (Da), 700000 daltons (Da), 800000 daltons (Da), 1000000 daltons (Da), 1500000 daltons (Da), 2000000 daltons (Da), 2500000 daltons (Da), but having a molecular weight of the hyaluronic acid molecules which is lower than 3000000 daltons (Da), 2500000 daltons (Da), 2225000 daltons (Da), 2000000 daltons (Da), 1750000 daltons (Da), 1500000 daltons (Da), 1250000 daltons (Da), 1000000 daltons (Da), 900000 daltons (Da), 800000 daltons (Da), 700000 daltons (Da), 600000 daltons (Da), 500000 daltons (Da), 100000 daltons (Da), 300000 daltons (Da), 200000 daltons (Da), 150000 daltons (Da) or 100000 daltons (Da).

In particular, the inventors have observed that these hyaluronic acid molecules having a high molecular weight could be used as a carrier for hyaluronic acid molecules of lower molecular weight, especially hyaluronic acid molecules of low molecular weight associated with a surfactant (molecule), but also as a moisture compound for an efficient hydration of the human skin.

Similarly, hyaluronic acid molecules having low molecular weight could be used as carrier for hyaluronic acid molecules of high molecular weight, especially these molecules associated with a surfactant. Usually, molecules which are used as carrier are molecules which are present in excess of molecules associated with the surfactant.

This composition does not need additional cosmetic or pharmaceutical excipients usually included in the composition of the state of the art.

Advantageously, the composition according to the invention does not present any side effects, such as allergy and is made of constituents which are considered as safe and secure for topical administration to mammals, especially to humans.

Preferably, the combination of a polyamine with hyaluronic acid allows the association (complexation) of particles being micels that result into a colloidal composition.

The composition could be a colloidal solution comprising particles having a size comprised between 10⁻⁶ m and 10⁻⁹ m.

A colloid is a fine state of subdivision of particles that are too small to be visible by an ordinary optical microscope and do not settle or settle slowly.

A solution is a solid liquid or gas mixed homogenously with a liquid being a solvent.

A solvent is a substance capable of using dissolving or dispersing one or more substances, especially a liquid component or a solution present in a great amount under the solute.

The colloidal composition according to the invention could be selected from the group consisting of a water in oil emulsion/oil in water emulsion, microemulsion, a clear solution, a suspension of nanoparticles or an anhydrous composition.

A suspension is made of particles mixed in a fluid or in a solid, but undissolved. An emulsion is an ultimate mixture of two incompletely miscible liquids, so one liquid is dispersed with or without an emulsion into the other liquid.

Preferably, polyamines are alkyl amines derivatives, such as diethylamine, dipropylamine, triethylamine, triethanolamine, tripropylamine, tripentylamine, trihexylamine, trioctylamine, tridodecylamine, or other amines with a longer chain length higher than 12, but preferably lower than 50, more preferably lower than 20.

Preferably, the above mentioned selected polyamines present in the composition according to the invention are aliphatic amines or heterocyclic (with one or more heteroatoms) amines comprising 1, 2, 3, 4, 5, 6, 7, 8, 9 or 10 carbons (preferably 5, 6, 7 or 8 carbons).

Other examples of preferred polyamines present in the composition according to the invention are endogenous polyamines, such as putrecine which is preferred, spermine or its precursor spermidine which is a nitrosamine and which may have some deleterious effects (toxic effect) the polylysine, the polyarginine and the polyhistidine, or other examples of natural or synthetic polyamines.

The cosmetic or pharmaceutical composition according to the invention could be also used as a carrier for other active cosmetic or pharmaceutical compound, especially vitamins (such as vitamin C, vitamin E, vitamins K1 or K2, vitamin K1 oxide or vitamin K2 oxide, etc).

Another aspect of the present invention is related to a method for the preparation of the composition according to the invention which comprises the steps of:

combining a polyamine (molecule) and hyaluronic acid molecule(s) having a high molecular weight, higher than 20000 daltons (Da), but lower than 3000000 daltons (Da), more preferably the above mentioned molecular weight of hyaluronic acid molecule to form a complex.

Preferably, the hyaluronic acid molecules are present in their salt form, preferably as sodium hyaluronate.

Furthermore, the composition formulation according to the invention could also comprise other compounds which are skin beneficial ingredients, preferably selected from the group consisting of skin cleansers, skin and hair conditioning agents, vitamins (vitamin C, vitamin E, vitamin K1 or K2, vitamin K1 oxide or vitamin K2 oxide, etc), hormones, minerals, plant extracts, anti-inflammatory agents, concentrates of plant extracts, emollients, moisturizers, skin protectants, humectants, silicones, skin soothing ingredients, analgesics, skin penetration enhancers, solubilizers, emollients, alkaloids, memory enhancers, anesthetics, colorants, perfumes, preservative, seeds, broken seed nut shells, silica, clays, beads, luffa particles, polyethylene balls, mica, pH adjusters, processing aids, and combinations thereof. The quantities of such ingredients can be safe and effective amounts as needed and not limited to any specific limits.

These various elements could be used in order to modify or improve the characteristics of the composition according to the invention.

The composition may also comprise the various known elements which improve the characteristics of a cosmetic or pharmaceutical composition. These elements are for example antioxidants, collagen and elastin synthetic boosters (such as L-tyrosine), various hydroxy acids (alpha hydroxyl acids, beta hydroxyl acids, polyhydroxy acids, vitamins, hormones, skin whitening agents, UVA/UVB sunscreens, antimicrobial agents, antifungal agents, blood microcirculation improvement agents (vasodilatory or vasoconstrictive), skin protectant drug actives, and combinations thereof. Such elements could also comprise elements which promote collagen and elastin in the skin, such as ascorbic acid, ascorbic acid derivatives, glucosamine ascorbate, arginine ascorbate, lysine or tyrosine ascorbate, gluthathione ascorbate, nicotinamide ascorbate, niacin ascorbate, allantoin ascorbate, creatine ascorbate, creatinine ascorbate, chondroitin ascorbate, chitosan ascorbate, DNA ascorbate, carnosine ascorbate, vitamin E, vitamin E derivatives, tocotrienol, rutin, quercetin, hesperedin, diosmin, mangiferin, mangostin, cyanidin, astaxanthin, lutein, lycopene, resveratrol, tetrahydrocurcumin, rosmarinic acid, hypericin, ellagic acid, chlorogenic acid, oleuropein, alpha-lipoic acid, niacinamide lipoate, gluthathione, andrographolide, carnosine, niacinamide, polyphenols, pycnogenol and combinations thereof.

Advantageously, the composition according to the invention could be selected to provide the treatment of a human skin, especially for an efficient hydattation of the skin, combined with an efficient therapeutically or cosmetic effect, skin wrinkles reduction, skin exfoliating, treatment of acne, treatment of rosacea, age-spots reduction, skin surface whitening, skin surface brightening, stria distensae (stretch marks), reduction treatment of pimples, treatment of skin infections and lesions, including surgery lesions, varicose and spider veins reduction, blood microcirculation improvement, UVA/UVB protection of skin, skin redness reduction benefits for the treatment of microbial or fungal contaminants, cellulite control, skin and body toning benefits or combinations thereof, preferably without inducing side effects such as allergy.

The present invention is related to the composition according to the invention for use as a medicament.

Another aspect of the present invention is related to the use of the composition according to the invention in the manufacture of a medicament for the treatment of wound healing, lesions, especially surgery lesions, skin infections, acne, varicose, spider veins and burns.

The composition according to the invention could be used as skin wrinkles filler or lips filler.

Another aspect of the present invention is related to a method of treating skin wrinkles (to obtain a reduction of skin wrinkles) preferably combined with a moisture effect and the skin which comprises the steps of administrating the composition upon the skin of a patient, in order to obtain efficient skin wrinkles reduction, skin wrinkles filling or lips filling.

The following table 1 present various absorbance results of different compositions comprising hyaluronic acid molecules and different types of a polyamine. These absorbance results are measured after various diffusion times (2 hours or 12 hours upon FRANZ cells at a constant hyaluronic acid concentration and with equivalent volumes of hyaluronic acid and of a polyamine.

TABLE 1

Time

| Absorbance results | 2 h | 12 h |
|---|---|---|
| Hyaluronic acid (1000000 Da) 2% | 0.01123 | 0.01415 |
| Hyaluronic acid + BDHAC | 0.0334 | 0.0516 |
| Hyaluronic acid + trioctylamine | 0.3212 | 0.4254 |
| Hyaluronic acid + 2-aminopyridine | 0.8324 | 1.4953 |
| Hyaluronic acid + spermine | 0.392 | 1.512 |
| Hyaluronic acid + spermidine | 0.9178 | 1.3481 |
| Hyaluronic acid + putrecine | 0.8704 | 1.4855 |

As described in table 1, putrecine is the preferred selected compound, because it presents a high absorbance after 2 and after 12 hours, because is obtained at low cost and because it is not toxic.

1. A cosmetic or pharmaceutical composition comprising particles of hyaluronic acid having a molecular weight comprised between 20000 Da and 3000000 Da, combined with a polyamine.

2. The composition according to the claim 1, wherein the hyaluronic acid particles have a molecular weight comprised between 50000 Da and 2000000 Da.

3. The composition according to the claim 1, wherein the hyaluronic acid particles have a molecular weight comprised between 100000 Da and 2000000 Da.

4. The composition according to claim 1, wherein the polyamine is putrecine.

5. The composition according to claim 1, wherein the particles of hyaluronic acid combined with polyamine are micels.

6. The composition according to claim 1 which further comprises a solvent.

7. The composition according to claim 1 which comprises molecules of hyaluronic acid having different molecular weight.

8. The composition according to claim 1, which is a skin wrinkles filler or a lips filler.

9. (canceled)

10. A method for the treatment and/or the prevention of a pathology selected from the group consisting of wounds healing, burns, skin surgery, skin aging, skin exfoliating, treatment of acne, treatment of rosacea, age-spots reduction, skin surface whitening, skin surface brightening, stria distensae (stretch marks), reduction treatment of pimples, treatment of skin infections and lesions, varicose and spider veins reduction, blood microcirculation improvement, UVA/UVB protection of skin, skin redness reduction benefits for the treatment of microbial or fungal contaminants, cellulite control, skin and body toning benefits or combination thereof, which comprises the step of administrating an efficient amount of the composition according to claim 1 to the patient.

11. The composition of claim 6, wherein the solvent is water or glycerine.
